(19) United States
(12) Patent Application Publication
(10) Pub. No.: US 2014/0088584 A1
(43) Pub. Date: Mar. 27, 2014

(54) MEDICAL DEVICE BALLOON CATHETER

(71) Applicant: BOSTON SCIENTIFIC SCIMED, INC., Maple Grove, MN (US)

(72) Inventors: ADAM J. ROYER, BROOKLYN PARK, MN (US); ROBERT N. SQUIRE, MAPLE GROVE, MN (US); CASS A. HANSON, ST. PAUL, MN (US); JEFFREY S. LINDQUIST, MAPLE GROVE, MN (US); DEREK C. SUTERMEISTER, HAM LAKE, MN (US)

(73) Assignee: BOSTON SCIENTIFIC SCIMED, INC., MAPLE GROVE, MN (US)

(21) Appl. No.: 14/037,091

(22) Filed: Sep. 25, 2013

Related U.S. Application Data
(60) Provisional application No. 61/705,968, filed on Sep. 26, 2012.

(51) Int. Cl. A61B 18/14; A61B 18/18

(52) U.S. Cl. CPC A61B 18/1492; A61B 18/18
USPC 606/33

(57) ABSTRACT

An intravascular catheter for tissue modulation, comprising an elongate member having a proximal end and a distal end, a balloon disposed on the elongate member and having an interior surface, an exterior surface, a lumen defined by the interior surface and at least a first region for transmitting therapeutic energy from the exterior surface of the balloon, wherein the elongate member has a fluid supply lumen having a plurality of ports fluidly connected to the balloon lumen and a fluid evacuation lumen having a port fluidly connected to the balloon lumen.

### CROSS-REFERENCE TO RELATED APPLICATIONS

This application claims priority under 35 U.S.C. §119 to U.S. Provisional Application Ser. No. 61/705,968, filed Sep. 26, 2012, the entirety of which is incorporated herein by reference.

### FIELD

The disclosure generally pertains to percutaneous and intravascular devices for nerve modulation and/or ablation.

BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many body tissues such as nerves, including renal nerves, brain tissue, cardiac tissue and the tissue of other body organs are in close proximity to blood vessels or other body cavities and thus can be accessed percutaneously or intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

In treatments involving perivascular nerves such as renal nerves, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, vessel thrombus, and/or protein fouling of the electrode.

### SUMMARY

It is therefore desirable to provide for alternative systems and methods for tissue treatment such as intravascular nerve modulation which distributes ablation or modulation sites along and around the vessel or other body cavity.

Some embodiments of the disclosure are directed to a balloon catheter configured for tissue modulation such as nerve modulation and/or ablation. The balloon catheter includes an inflatable balloon at or proximate a distal end of the device. The wall of the balloon is constructed so as to include areas for transmitting therapeutic energy from the balloon into body tissue. These areas may be, for example, RF electrodes located on the surface of the balloon or ionically permeable windows for permitting the transmission of ionic energy from within the balloon lumen. Fluid may be supplied to the balloon through a plurality of fluid inlet ports and may be evacuated from the balloon through one (or more) fluid outlet ports. The plurality of fluid inlet ports direct the inflation fluid against the interior of the balloon wall. The plurality of fluid inlet ports may be configured to direct the fluid directly against the areas for transmitting therapeutic energy or may be configured to direct the fluid against areas of the interior of the balloon wall other than those areas for transmitting therapeutic energy.

The above summary of some example embodiments is not intended to describe each disclosed embodiment or every implementation of the disclosure.

### BRIEF DESCRIPTION OF DRAWINGS

The disclosure may be more completely understood in consideration of the following detailed description of various embodiments in connection with the accompanying drawings, in which:

FIG. 1 is a schematic view illustrating a renal nerve modulation system in situ.

FIG. 2 is a schematic view illustrating the distal end of a renal nerve modulation system.

FIG. 3 is a cross-sectional view of the renal nerve modulation system of FIG. 2.

FIG. 4 is a cross-sectional view of the renal nerve modulation system of FIG. 2.

FIG. 5 is a schematic view illustrating the distal end of a renal nerve modulation system.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless clearly stated to the contrary.

While the devices and methods described herein are discussed relative to renal nerve modulation through a blood vessel wall, it is contemplated that the devices and methods may be used in other applications where nerve modulation and/or ablation are desired. The term modulation refers to ablation and other techniques that may permanently alter the function of affected nerves and other tissue such as brain tissue or cardiac tissue. When multiple ablations are desirable, they may be performed sequentially by a single ablation device. In some embodiments, one ablation catheter can perform multiple treatments.

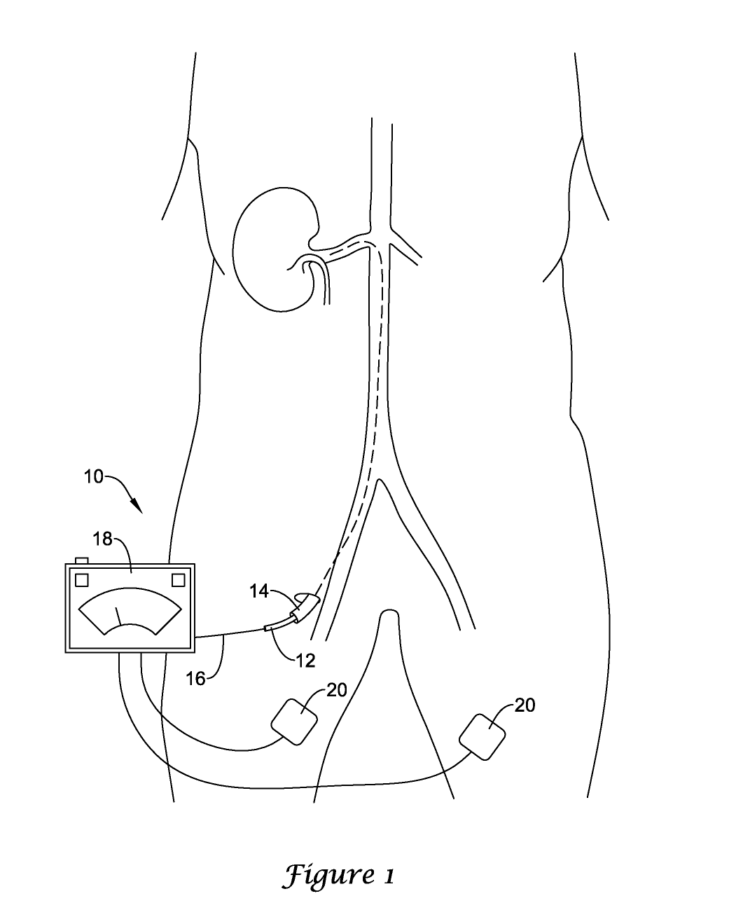

FIG. 1 is a schematic view of an illustrative renal nerve modulation system in situ. System 10 may include one or more conductive element(s) 16 for providing power to a renal ablation system including a renal nerve modulation device 12 disposed within a delivery sheath 14, which may be adapted to slidably contain the renal nerve modulation device 12 when the radially expanding region (not shown) of the elongate member is in a non-expanded configuration, the details of which can be better seen in subsequent figures. A proximal end of conductive element(s) 16 may be connected to a control and power element 18, which supplies necessary electrical energy to activate one or more electrodes to which the distal end of wire(s) 16 are attached at or near a distal end of the renal nerve modulation device 12. When suitably activated, the electrodes are capable of ablating tissue as described below. The terms electrode and electrodes may be considered to be equivalent to elements capable of ablating adjacent tissue in the disclosure which follows. Suitable materials for the delivery sheath 14, renal nerve modulation device 12 and elements capable of ablating adjacent tissue are known in the art and in some embodiments may include internal and/or external layers of lubricious material(s). In some instances, return electrode patches 20 may be supplied on the legs, the back near the kidneys or at another conventional location on the patient's body to complete the circuit. A proximal hub (not illustrated) having ports for a guidewire, an inflation lumen and a return lumen may also be included.

The control and power element 18 may include monitoring elements to monitor and control parameters such as power, temperature, time, voltage, pulse size, impedance and/or shape and other suitable parameters, with sensors mounted along renal nerve modulation device 12, as well as suitable controls for performing the desired procedure. In some embodiments, the power element 18 may control a radio frequency (RF) electrode. The electrode may be configured to operate at a frequency of approximately 460 kHz. It is contemplated that any desired frequency in the RF range may be used, for example, from 450-500 kHz. It is further contemplated that other ablation devices may be used as desired, for example, but not limited to resistance heating, ultrasound, microwave, and laser devices and these devices may require that power be supplied by the power element 18 in a different form.

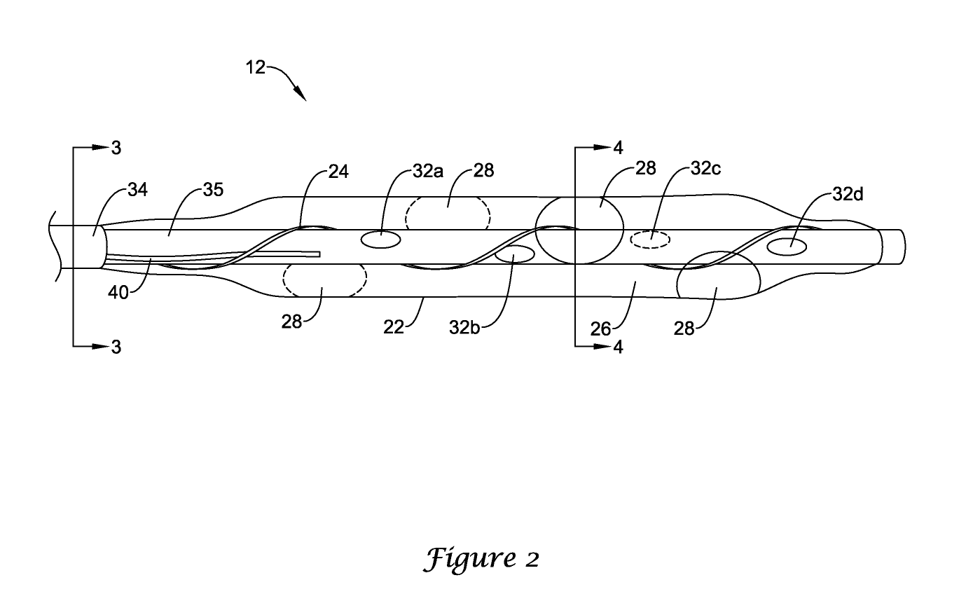

FIG. 2 illustrates the distal portion of a renal nerve modulation device 12. Renal nerve modulation device 12 includes a balloon 22 and an electrode (or transmitter) 24. When in use, the balloon is preferably filled with a conductive fluid 26 such as saline to allow the ablation energy to be transmitted from the electrode 24 through windows 28 that are permeable to ionic energy. The windows 28 are arranged to achieve complete circumferential coverage of the blood vessel while being spaced apart longitudinally. Other appropriate conductive fluids include hypertonic solutions, contrast solution and mixtures of saline or hypertonic saline solutions with contrast solutions. The conductive fluid may be introduced through fluid inlets 32a-32d in a central shaft 35 and evacuated through a fluid outlet between central shaft 35 and an outer shaft 34. One or more sensors 40, such as a thermocouple, may be included and may be disposed on the shaft 34, on the balloon 22 or at another suitable location. Further details may be had with reference to U.S. Patent Provisional Application No. 61/605,615, filed Mar. 1, 2012 and titled “DEVICES AND METHODS FOR NERVE MODULATION USING A NOVEL CATHETER WITH POLYMERIC ABLATION ELEMENTS,” incorporated herein by reference.

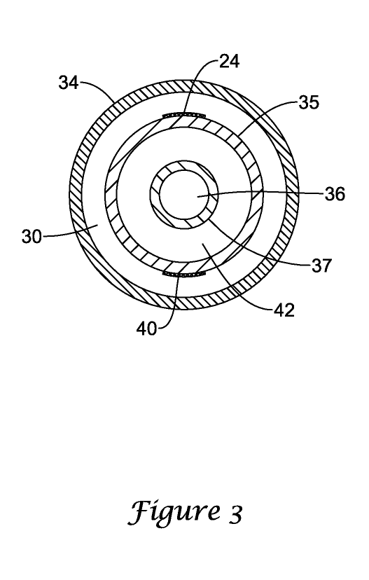

A cross-sectional view of the central shaft 35 and outer shaft 34 at section line 3 in FIG. 2 is illustrated in FIG. 3. The central shaft 35 may include a guidewire tube 37 defining a guidewire lumen 36. The central shaft 35 includes a fluid inlet lumen 42 that fluidly connects to the fluid inlets 32a-32d. A fluid outlet lumen 30 is defined by the outer shaft 34 and the inner shaft 35. The electrode 24, or a conductive element to supply power to the electrode may extend along the outer surface of the shaft 35 or may be embedded within the shaft 35. The electrode 24 proximal to the balloon is preferably electrically insulated and is used to transmit power to the portion of the electrode disposed in the balloon. The sensor 40 may also extend along the shaft 35. The shafts may be generally concentrically arranged as illustrated or some other suitable arrangement may be used. For example, the lumens 42, 30 of central shaft 35 may have a side-by-side arrangement or an embodiment may include a single multi-lumen shaft where the fluid inlet lumen, fluid outlet lumen and guidewire lumens are incorporated into a single shaft. Some embodiments may omit the guidewire lumen or include other elements such as steering wires and the like. The lumens 30, 42 may extend proximally to a hub where they may be connected to an evacuation reservoir or vacuum, and a fluid supply or pump, respectively.

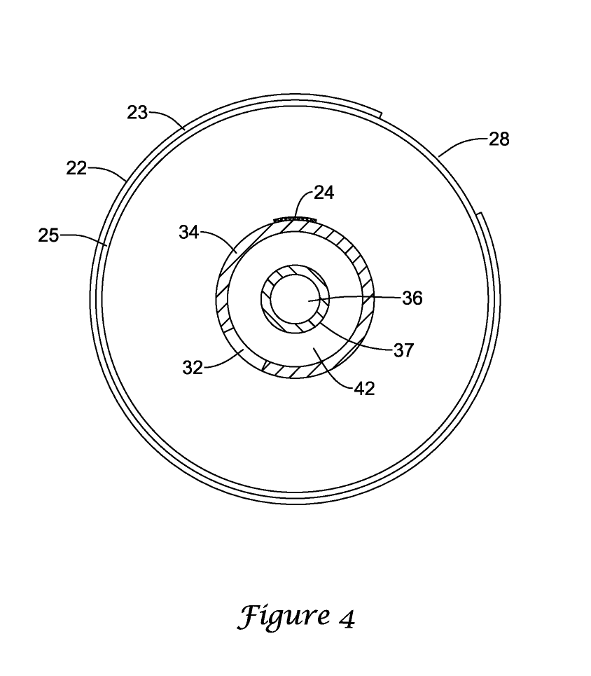

A cross-sectional view of the shaft 34 of the renal nerve modulation device 12 at section line 4 in FIG. 2 is illustrated in FIG. 4. Shaft 35 may include a guidewire lumen 36 and a lumen 42 connected to the fluid inlet port 32. In some embodiments, the guidewire lumen extends from the distal end of the device to a proximal hub. In other embodiments, the guidewire lumen can have a proximal opening that is distal to the proximal portion of the system.

Balloon 22 is shown in cross-section as having a first layer 25 and a second layer 23. A window 28 is formed in balloon 22 by the absence of second layer 23. First layer 25 is preferably made from an ionically permeable material. One suitable material is, for example, a hydrophilic polyurethane. The second layer 23 is preferably made from an electrically non-ionically permeable polymer such as a non-hydrophilic polyurethane, Pebax, nylon, polyester or block-copolymer.

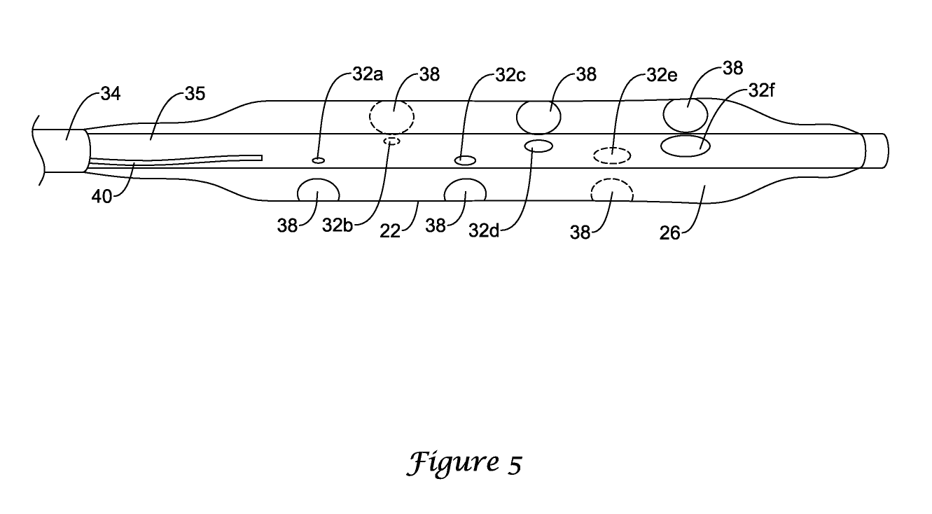

In the embodiment illustrated in FIGS. 2 and 5, the ionic energy from the electrode 24 may become concentrated (higher current density) at the edges of the conductive windows where there is a large delta in impedance of the two adjacent materials (window material and insulation coating material) of the balloon 22 and in particular in the portions of the balloon wall that lack the non-conductive second layer 23. Thus ports 32 are located and configured to direct the fluid against those regions of the balloon wall. For example, it can be seen in both FIGS. 2 and 3 that the ports 32 are directed at the balloon windows 28 under standard flow conditions. These ports may be configured to direct the fluid directly out of the port or may be configured to direct the fluid at an angle. “At an angle” herein means at a non-zero angle to a radius of the balloon. For examples, the ports 32 may direct the fluid at an angle such that the fluid is given a clockwise or counterclockwise motion in the balloon lumen. The ports 32 may, alternatively or in addition, be directed to angle the fluid in a proximal direction. The ports 32 may all be at the same angle or may be at different angles relative to each other. The ports 32 may be the same size or different sizes.

FIG. 5 illustrates the distal portion of a renal nerve modulation device 12 that includes a balloon 22 on which are disposed RF electrodes 38. Electrodes 38, disposed on the balloon 22, are arranged to achieve complete circumferential coverage of the blood vessel while spaced apart longitudinally and may be powered by leads (not illustrated) extending along the surface of the balloon or extending up from the central shaft 35. The electrodes 38 are depicted as circular but may be any desired shape. They may, for example, be oval or oblong. The balloon 22 is filled through a fluid supply lumen through ports 32a-32f. The fluid is evacuated through a fluid evacuation lumen connected to fluid outlet lumen 30. One port 32 is provided under, or adjacent to, each electrode 38. Each one of the ports 32 may be configured to direct the fluid directly out of the port towards the respective electrode 38 or may be configured to direct the fluid at an angle towards the respective electrode 38. Ports 32 are illustrated as having different sizes with the proximal-most port, port 32a, being the smallest and the distal-most port, port 32f, being the largest. The size of the ports may vary to maintain a uniform output of fluid from each port or may be varied to provided more or less fluid flow against particular areas of the balloon and/or or particular electrodes. Other variations of the ports such as discussed above may be included as well.

In use, a renal ablation system such as system 10 is provided. The system may be used with a standard guide catheter such as a 6 French guide catheter. The balloon and in particular the hydrophilic or tecophilic material may be hydrated as part of the preparatory steps. Hydration may be effected by soaking the balloon in a saline solution. A one minute, five minute, or other suitable soak may be beneficial. Then the renal nerve modulation device 12 may then be introduced percutaneously as is conventional in the intravascular medical device arts by using a guide catheter and/or a guide wire. For example, a guide wire such as a 0.014″ diameter guidewire may be introduced percutaneously through a femoral artery and navigated to a renal artery using standard radiographic techniques. In some embodiments, a delivery sheath 14 may be introduced over the guide wire and the guide wire may be withdrawn, and the renal nerve modulation device 12 may be then introduced through the delivery sheath. In other embodiments, the renal nerve modulation device 12 may be introduced over the guidewire, or the system 10, including a delivery sheath 14 may be introduced over a guidewire. In embodiments involving a delivery sheath 14, the renal nerve modulation device 12 may be delivered distally from the distal end of the delivery sheath 14 into position, or the delivery sheath may be withdrawn proximally to expose the distal portion of renal nerve modulation device 12. A conductive fluid 26 is introduced into the balloon through fluid inlet ports 32 and may expand the balloon to the desired size. The balloon expansion may be monitored indirectly by monitoring the volume, or flow rate, of conductive fluid introduced into the system or may be monitored through radiographic or other conventional means. Once the balloon is expanded to the desired size, the conductive fluid 26 may be circulated within the balloon by continuing to introduce the fluid through the fluid inlet ports 32 while withdrawing fluid from the balloon through the fluid outlet 30. The rate of circulation of the fluid may be between 2 and 20 ml/min, between 3 and 15 ml/min, between 5 and 75 ml/min or other desired rate of circulation.

The balloon may be kept at or near a desired pressure such as a pressure of between 0.25 and 6 atmospheres, between 1.5 and 4 atmospheres, between 2.5 and 3.5 atmospheres, or other desired pressure. The electrode(s) is then activated by supplying energy. The energy may be supplied at 400-500 Hz and at between 0.05 and 1 amp. The energy is transmitted to the blood vessel wall to modulate or ablate the surrounding tissue. The progress of the treatment may be monitored by monitoring changes in impedance through the electrode. Other measurements such as pressure and/or temperature measurements may be conducted during the procedure as desired. The circulation of the conductive fluid 26 may mitigate the temperature rise of the tissue or the blood vessel 48 in contact with the windows 28. The electrode 24 is preferably activated for an effective length of time, such as 1 minute or 2 minutes. Once the procedure is finished at a particular location, the balloon 22 may be partially or wholly deflated and moved to a different location such as the other renal artery, and the procedure may be repeated at another location as desired using conventional delivery and repositioning techniques. Various modifications and alterations of this disclosure will become apparent to those skilled in the art without departing from the scope and principles of this disclosure, and it should be understood that this disclosure is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An intravascular catheter for tissue modulation, comprising:
an elongate member having a proximal end and a distal end; and
a balloon disposed on the elongate member and having an interior surface, an exterior surface, a lumen defined by the interior surface and at least a first region for transmitting therapeutic energy from the exterior surface of the balloon,
wherein the elongate member has a fluid supply lumen having a plurality of ports fluidly connected to the balloon lumen and a fluid evacuation lumen having a port fluidly connected to the balloon lumen.

2. The intravascular catheter of claim 1, wherein the port of the fluid evacuation lumen is disposed proximal of at least one of the plurality of ports of the fluid supply lumen.

3. The intravascular catheter of claim 1, wherein the port of the fluid evacuation lumen is disposed proximal of all of the plurality of ports of the fluid supply lumen.

4. The intravascular catheter of claim 1, wherein the balloon comprises a plurality of regions for transmitting therapeutic energy, and wherein one of the plurality of ports of the fluid supply lumen is disposed directly under one of the plurality of regions for transmitting therapeutic energy.

5. The intravascular catheter of claim 1, wherein at least one of the plurality of ports of the fluid supply lumen is designed to direct fluid at an angle that is substantially normal to a longitudinal axis of the elongate member.

6. The intravascular catheter of claim 1, wherein at least one of the plurality of ports of the fluid supply lumen is designed to direct fluid at an angle relative to a longitudinal axis of the elongate member.

7. The intravascular catheter of claim 1, wherein at least one of the plurality of ports of the fluid supply lumen is designed to direct fluid proximally.

8. The intravascular catheter of claim 1, wherein at least one of the plurality of ports of the fluid supply lumen is designed to direct fluid distally.

9. The intravascular catheter of claim 1, wherein the plurality of ports of the fluid supply lumen are designed to direct fluid clockwise or counterclockwise within the balloon lumen.

10. The intravascular catheter of claim 1, wherein the plurality of ports of the fluid supply lumen are all the same size.

11. The intravascular catheter of claim 1, wherein at least some of the plurality of ports of the fluid supply lumen differ in size.

12. The intravascular catheter of claim 11, wherein the plurality of ports of the fluid supply lumen increase in size distally along the elongate member.

13. The intravascular catheter of claim 1, wherein the first region for transmitting therapeutic energy includes an RF electrode coupled to the balloon.

14. The intravascular catheter of claim 1, wherein an electrode is disposed within the balloon lumen, wherein the first region for transmitting therapeutic energy includes an ionically permeable region disposed along the balloon.

15. A medical device for nerve modulation, comprising:
a catheter shaft having a distal end region, the catheter shaft including an inner tubular member and an outer tubular member;
a balloon coupled to the distal end region, the balloon including one or more energy transmitting regions;
wherein the shaft defines a fluid supply lumen and a fluid return lumen;
wherein the shaft includes a plurality of fluid supply ports disposed within the balloon and in fluid communication with the fluid return lumen; and
an electrode coupled to the shaft.

16. The medical device of claim 15, wherein the fluid supply lumen is defined within the inner tubular member.

17. The medical device of claim 15, wherein the fluid return lumen is defined between the inner tubular member and the outer tubular member.

18. The medical device of claim 15, wherein the plurality of fluid supply ports are defined in the inner tubular member and wherein the plurality of fluid supply ports get progressively larger distally along the inner tubular member.

19. The medical device of claim 15, further comprising one or more sensors disposed adjacent to the balloon.

20. A method of nerve modulation, comprising:
providing a medical device, the medical device comprising:
a catheter shaft having a distal end region, the catheter shaft including an inner tubular member and an outer tubular member,
a balloon coupled to the distal end region, the balloon including one or more energy transmitting regions,
wherein the shaft defines a fluid supply lumen and a fluid return lumen,
wherein the shaft includes a plurality of fluid supply ports disposed within the balloon and in fluid communication with the fluid return lumen, and
an electrode coupled to the shaft;
advancing the medical device through a blood vessel to a position adjacent to an area of interest;
inflating the balloon; and
transmitting therapeutic energy from the one or more energy transmitting regions.
